(19) United States
(12) Patent Application Publication
(10) Pub. No.: US 2025/0362287 A1
(43) Pub. Date: Nov. 27, 2025

(54) METHODS OF SCREENING FOR PEPTIDE-HLA CLASS I ALLOREACTIVITY

(71) Applicant: AMGEN INC., Thousand Oaks, CA (US)

(72) Inventors: Ana S. GOYOS, Thousand Oaks, CA (US); Mozhgan FARSHBAF, Thousand Oaks, CA (US); Kavita RAMAN, Thousand Oaks, CA (US); Ian FOLTZ, Thousand Oaks, CA (US)

(73) Assignee: AMGEN Inc., Thousand Oaks, CA (US)

(21) Appl. No.: 18/872,187

(22) PCT Filed: Jun. 6, 2023
(86) PCT No.: PCT/US2023/024600

§ 371 Date: Dec. 5, 2024

Related U.S. Application Data
(60) Provisional application No. 63/350,622, filed on Jun. 9, 2022.

(51) Int. Cl. G01N 33/50

(52) U.S. Cl. CPC G01N 33/5023; G01N 33/505

(57) ABSTRACT

Provided herein is a recombinant cell comprising a deletion of a gene encoding a transporter associated with antigen processing (TAP) protein and mutations in the CD3 epsilon gene and an HLA-A gene. Also provided are systems and methods for screening for alloreactivity and specificity of an immunotherapeutic agent, such as a bispecific T cell engager or an engineered T cell receptor (TCR).

### CROSS-REFERENCE TO RELATED APPLICATIONS

This application claims the benefit of U.S. Provisional Patent Application No. 63/350,622, filed Jun. 9, 2022, which is hereby incorporated by reference in its entirety.

### FIELD

The disclosure is directed to systems and methods for assessing alloreactivity of an immunotherapeutic agent using a recombinant cell line.

### BACKGROUND

Immunotherapeutic approaches for the treatment of cancer exploit the potent cytotoxic properties of T-cells. Immunotherapeutic products, such as bispecific T cell engager (BiTE®) molecules and engineered T cell receptors (TCRs) can target intracellular antigens not normally accessible on the cell surface. The human-specific nature of the genes coding these target proteins and immunotherapeutic modalities precludes the use of animal models for nonclinical safety assessment. Instead, such safety assessments rely on a diverse array of in vitro and in silico tools to properly de-risk these modalities and targets.

T lymphocytes recognize antigen in the form of peptide bound to major histocompatibility complex molecules (pMHC). Antigen recognition in the context of self-MHC by a T cell receptor (TCR) plays an important part in the development of T cells in the thymus. In the context of certain types of immunotherapeutics, such as BiTE® molecules and engineered TCRs, alloreactivity may occur when the immunotherapeutic cross-reacts with an off-target peptide complexed to a compatible or non-target HLA allele. One important component of de-risking pMHC targeting modalities involves an assessment of HLA class I allogeneic cross-reactivity. Standard practice involves screening against a panel of B lymphoblastoid cell lines representing HLA class I allotypic diversity of the intended patient population. This approach, however, is dependent on the B cell-specific peptide repertoire to help uncover HLA alloreactivity.

There remains a need for more controllable systems and methods to assess both HLA alloreactivity and peptide specificity of immunotherapeutic agents.

### BRIEF SUMMARY

The disclosure provides recombinant cell comprising: (a) a deletion of a gene encoding the transporter associated with antigen processing (TAP) protein: (b) at least one genome mutation in a CD3ε gene; and (c) at least one genome mutation in an HLA-A gene.

In some aspects, the recombinant cell does not express a TAP gene, the CD3ε gene, and the HLA-A gene. In some aspects, the HLA-A gene comprises an HLA-A*02:01 allele.

In some aspects, the recombinant cell is a hybrid T and B lymphoblastoid cell, such as a T2 cell.

In some aspects, the recombinant cell expresses a luciferase gene.

In other aspects, the recombinant cell comprises an exogenous nucleic acid sequence encoding an HLA-A allele, such as, e.g., A*02:01:01:01, A*02:02:01:01, A*02:03:01, A*02:05:01:01, A*02:06:01:01, A*02:07:01:01, A*02:11:01:01, A*01:01:01:01, A*03:01:01:01, A*11:01:01:01, A*23:01:01:01, A*24:02:01:01, A*30:01:01:01, A*31:01:02:01, A*33:03:01:01, A*68:01:01:01, A*68:02:01:01, A*69:01:01:01, or A*74:01:01:01.

The disclosure also provides a library comprising a plurality of the aforementioned recombinant cells, wherein each cell comprises an exogenous nucleic acid sequence encoding a different HLA-A allele.

The disclosure further provides a system comprising: (a) the aforementioned library of recombinant cells: (b) one or more peptides: (c) one or more immunotherapeutic agents; and (d) one or more T cells.

In some aspects of the disclosed system, the one or more peptides comprise one or more cancer antigens, such as, e.g., a MAGE peptide, a BCMA peptide, a CD19 peptide, a CD33 peptide, a DLL3 peptide, a FLT3 peptide, a MUC17 peptide, a PSMA peptide, or a CLDN18.2 peptide.

In other aspects of the disclosed system, the one or more immunotherapeutic agents comprise an engineered T cell receptor (TCR) or a bi-specific T cell engager protein.

In some aspects of the disclosed system, the T cells are effector T cells.

The disclosure further provides a method for determining alloreactivity of an immunotherapeutic agent, which method comprises (a) contacting the aforementioned library of recombinant cells with one or more peptides, one or more immunotherapeutic agents, and one or more T cells, whereby the one or more peptides are presented at the surface of the recombinant cell bound to one or more major histocompatibility complex (MHC) molecules (pMHC); and (b) assessing cytotoxicity of the recombinant cells, wherein increased cytotoxicity as compared to control cells indicates alloreactivity of the immunotherapeutic agent.

Also provided is a method for determining binding specificity between a peptide and an immunotherapeutic agent, which method comprises: (a) contacting the aforementioned library of recombinant cells with one or more peptides, one or more immunotherapeutic agents, and one or more T cells, whereby the one or more peptides are presented at the surface of the recombinant cell bound to one or more major histocompatibility complex (MHC) molecules (pMHC); and (b) assessing cytotoxicity of the recombinant cells, wherein increased cytotoxicity as compared to control cells indicates that the immunotherapeutic agent specifically binds to the peptide.

In some aspects of the disclosed methods, the one or more peptides comprise one or more tumor antigens, such as, e.g., a MAGE peptide, a BCMA peptide, a CD19 peptide, a CD33 peptide, a DLL3 peptide, a FLT3 peptide, a MUC17 peptide, a PSMA peptide, or a CLDN18.2 peptide.

In some aspects of the disclosed methods, the immunotherapeutic agent comprises an engineered T cell receptor (TCR) or a bispecific T cell engager.

In other aspects of the disclosed methods, the T cells are effector T cells.

In some aspects of the disclosed methods, the control cells comprise at least one mutation in a CD3ε gene and at least one mutation in an HLA-A gene and lack an exogenous nucleic acid sequence encoding an HLA-A allele. In some aspects, the control cells are T2 cells. In other aspects, the control cells express a luciferase gene.

In some aspects of the disclosed methods, cytotoxicity is assessed by performing a T cell dependent cellular cytotoxicity (TDCC) assay, such as a luciferase-based assay.

### BRIEF DESCRIPTION OF THE DRAWING(S)

FIGS. 1A-1C are graphs illustrating that parental T2 cells upregulate HLA-A*02:01 in a dose-responsive manner when pulsed with MAGE-B2 (FIG. 1A), SLK (FIG. 1B), and AFP peptides (FIG. 1C).

FIG. 2A is a graph illustrating successful knockout of CD3e and HLA-A*02:01 confirmed at the cell surface protein level. FIG. 2B is a graph illustrating that T2-Luc KO cells no longer upregulate HLA-A*02:01 when pulsed with peptide.

FIG. 3A is a graph demonstrating that high viability and luciferase activity are retained in T2-Luc HLA-A*02:01/CD3e double knockout (KO) cells. FIG. 3B is a graph demonstrating HLA-A*02:01 phenotype rescue in T2-Luc HLA-A*02:01/CD3e double KO cells after retroviral transduction with an HLA-A*02:01 construct.

FIG. 4 is a schematic diagram illustrating the methods for generating the disclosed recombinant cells and cell libraries, as well as methods for testing alloreactivity and specificity of immunotherapeutic agents using the disclosed cells and libraries.

### DETAILED DESCRIPTION

The present disclosure is predicated, at least in part, on the development of a screening platform to assess both HLA and peptide specificity of immunotherapeutic modalities such as BiTE© (bispecific T cell engagers) and engineered T cell receptors (TCRs). This platform involves the genomic engineering of a cell line incapable of presenting endogenous peptides to express HLA class I allotypes of interest, to generate a library of single HLA-A allotype-expressing cell lines that can be used to assess cross-reactivity of peptide-HLA (pHLA) targeting modalities.

### Definitions

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

The term “alloreactivity,” as used herein, refers to cross-reactivity of an immunotherapeutic agent (e.g., BiTE® molecules, engineered TCRs, and chimeric antigen receptors (CARs)) with a non-target (also referred to as “off-target”) peptide complexed to a compatible or non-target HLA allele.

The term “recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may act to modulate production of a desired product by various mechanisms. Alternatively, DNA sequences encoding RNA that is not translated may also be considered recombinant. Thus, the term “recombinant” nucleic acid also refers to a nucleic acid which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid.

Alternatively, the artificial combination may be performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may comprise a naturally occurring amino acid sequence.

The terms “immunogen” and “antigen” may be used interchangeably herein and refer to any molecule, compound, or substance that induces an immune response in an animal (e.g., a mammal). An “immune response” can entail, for example, antibody production and/or the activation of immune effector cells. An antigen in the context of the disclosure can comprise any subunit, fragment, or epitope of any proteinaceous or non-proteinaceous (e.g., carbohydrate or lipid) molecule that provokes an immune response in a mammal.

The term “epitope” refers to any polypeptide determinant capable of specifically binding to an immunoglobulin, a T cell or B cell receptor, or any interacting protein, such as a surface protein. Epitopes also are referred to in the art as “antigenic determinants.” In certain embodiments, an epitope is a region of an antigen that is specifically bound by an antibody. In certain embodiments, an epitope may include chemically active surface groupings of molecules such as amino acids, sugar side chains, phosphoryl, or sulfonyl groups. In certain embodiments, an epitope may have specific three-dimensional structural characteristics (e.g., a “conformational” epitope) and/or specific charge characteristics.

The term “antigen-binding protein,” as used herein, refers to a proteinaceous molecule that specifically binds to an antigen. For example, an antigen-binding protein may be an antibody or an antigen-binding fragment thereof. An antigen-binding protein typically comprises the heavy chain variable region (VH) and/or the light chain variable region (VL) of an antibody, or comprises domains derived therefrom. In some embodiments, an antigen-binding protein comprises the minimum structural requirements of an antibody which allow for immunospecific target binding. This minimum requirement may be defined by, for example, the presence of at least three light chain complementarity determining regions (CDRs) (i.e., CDR1, CDR2 and CDR3 of the VL region) and/or three heavy chain CDRs (i.e., CDR1, CDR2 and CDR3 of the VH region), and preferably of all six CDRs. It is within the knowledge of a skilled person where (and in which order) those CDRs are located in the antigen-binding protein.

As used herein, the term “antibody” refers to a whole antibody molecule or a fragment thereof (e.g., fragments such as scFv, Fab, Fab′, and F(ab′)2), unless specified otherwise; an antibody may be a polyclonal or monoclonal antibody, a chimeric antibody, a humanized antibody, a human antibody, etc. In a native antibody, a heavy chain comprises a variable region, VH, and three constant regions, CH1, CH2, and CH3. The VH domain is at the amino-terminus of the heavy chain, and the CH3 domain is at the carboxy-terminus. In a native antibody, a light chain comprises a variable region, VL, and a constant region, CL. The variable region of the light chain is at the amino-terminus of the light chain. In a native antibody, the variable regions of each light/heavy chain pair typically form the antigen-binding site. The constant regions are typically responsible for effector function.

In a native antibody, the variable regions typically exhibit the same general structure in which relatively conserved framework regions (FRs) are joined by three hypervariable regions, also called complementarity determining regions (CDRs). The CDRs from the two chains of each pair typically are aligned by the framework regions, which may enable binding to a specific epitope. From N-terminus to C-terminus, both light and heavy chain variable regions typically comprise the domains FR1, CDR1, FR2, CDR2, FR3, CDR3 and FR4. The CDRs on the heavy chain are referred to as H1, H2, and H3, while the CDRs on the light chain are referred to as L1, L2, and L3. Typically, CDR3 is the greatest source of molecular diversity within the antigen binding site. The assignment of amino acids to each domain is typically in accordance with the definitions of Kabat et al. (1991) Sequences of Proteins of Immunological Interest (National Institutes of Health, Publication No. 91-3242, vols. 1-3, Bethesda, Md.); Chothia, C., and Lesk, A. M. (1987) J. Mol. Biol., 196: 901-917; or Chothia C. et al., Nature, 342:878-883 (1989). In the present application, the term “CDR” refers to a CDR from either the light or heavy chain, unless otherwise specified.

As used herein, when an antibody or other entity (e.g., antigen-binding domain) “specifically recognizes,” “specifically binds,” or “immunospecifically binds” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸ M⁻¹, >10⁹ M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

As used herein, the term “antibody fragment” refers to a portion of a full-length antibody, including at least a portion of an antigen-binding region or a variable region. Antibody fragments include, but are not limited to, Fab, Fab′, F(ab′)2, Fv, scFv, Fd, diabodies, and other antibody fragments that retain at least a portion of the variable region of an intact antibody (see. e.g., Hudson et al., Nat. Med., 9: 129-134 (2003)). In certain embodiments, antibody fragments are produced by enzymatic or chemical cleavage of intact antibodies (e.g., papain digestion and pepsin digestion of antibody) produced by recombinant DNA techniques, or by chemical polypeptide synthesis.

For example, a “Fab” fragment comprises one light chain and the CHT and variable region of one heavy chain. The heavy chain of a Fab molecule cannot form a disulfide bond with another heavy chain molecule. A “Fab′” fragment comprises one light chain and one heavy chain that comprises additional constant region, extending between the CH1 and CH2 domains. An interchain disulfide bond can be formed between two heavy chains of a Fab′ fragment to form a “F(ab′)2” molecule. An “Fv” fragment comprises the variable regions from both the heavy and light chains, but lacks the constant regions. A single-chain Fv (scFv) fragment comprises heavy and light chain variable regions connected by a flexible linker to form a single polypeptide chain with an antigen-binding region. Exemplary single chain antibodies are discussed in detail in WO 88/01649 and U.S. Pat. Nos. 4,946,778 and 5,260,203. In certain instances, a single variable region (e.g., a heavy chain variable region or a light chain variable region) may have the ability to recognize and bind antigen. Other antibody fragments will be understood by those of ordinary skill in the art.

“Nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to methylated and/or capped polynucleotides.

The term “genome,” as used herein, refers the complete set complete set of genes or genetic material present in a cell or organism.

The term “polypeptide” as used herein describes a group of molecules, which usually consist of more than 30 amino acids. Polypeptides may further form multimers such as dimers, trimers and higher oligomers, i.e., consisting of more than one polypeptide molecule. Polypeptide molecules forming such dimers, trimers, etc., may be identical or non-identical. The corresponding higher order structures of such multimers are, consequently, termed homo- or heterodimers, homo- or heterotrimers, etc. The terms “peptide”, “polypeptide” and “protein” also refer to naturally modified peptides/polypeptides/proteins wherein the modification is effected by, e.g., post-translational modifications like glycosylation, acetylation, phosphorylation and the like. A “peptide”, “polypeptide” or “protein” when referred to herein may also be chemically modified such as pegylated. Such modifications are well known in the art. A “peptide” generally is smaller than a protein or polypeptide, and typically comprises 2 to 50 amino acids.

The term “mutation,” as used herein, refers to the modification at least one physical feature of a wild-type DNA sequence of interest. A mutation is a permanent and heritable change in genetic material, which can result in altered protein function and phenotypic changes. Mutations include, for example, single or double strand DNA breaks, deletion or insertion of one or more nucleotides, and other modifications that affect the structural integrity or nucleotide sequence of the DNA sequence.

The terms “gene editing” and “genome editing” may be used interchangeably herein to refer to a type of genetic engineering in which DNA is inserted, deleted, modified, or replaced in the genome of a living organism. For example, gene editing may be used to disrupt or modify an endogenous genomic region of a host cell, inserting an exogenous gene into a host genome, replacing an endogenous nucleotide sequence with an exogenous nucleotide sequence, or any combination thereof. Systems and methods for gene editing are described in detail in, e.g., Doudna J A, Nature, 578(7794):229-236 (2020) doi: 10.1038/s41586-020-1978-5. Epub 2020; Khan, S. H, Molecular Therapy—Nucleic Acids, 16: 326-334 (2019); and National Academies of Sciences, Engineering, and Medicine; National Academy of Medicine; National Academy of Sciences; Committee on Human Gene Editing: Scientific, Medical, and Ethical Considerations. Human Genome Editing: Science, Ethics, and Governance. Washington (DC): National Academies Press (US); 2017 Feb. 14. A, The Basic Science of Genome Editing.

The term “bispecific,” as used herein, refers to an antigen-binding protein (e.g., an antibody) which is “at least bispecific,” i.e., it comprises at least a first binding domain and a second binding domain, wherein the first binding domain binds to one antigen or target (e.g., a target cell surface antigen), and the second binding domain binds to another antigen or target (e.g., a T cell activating domain). Accordingly, certain immunotherapeutic agents described herein comprise specificities for at least two different antigens or targets. The term “target cell surface antigen” refers to an antigenic structure expressed by a cell and which is present at the cell surface such that it is accessible for a bispecific protein. It may be a protein, preferably the extracellular portion of a protein, or a carbohydrate structure, preferably a carbohydrate structure of a protein, such as a glycoprotein. It is preferably a tumor antigen. The term “bispecific antigen-binding protein” also encompasses multispecific antigen-binding proteins such as trispecific antibodies, the latter ones including three binding domains, or antigen-binding proteins having more than three (e.g., four, five . . . ) specificities.

Given that the antigen-binding proteins according to the disclosure are (at least) bispecific, they do not occur naturally and they are markedly different from naturally occurring products. A “bispecific” antigen-binding protein or construct is hence an artificial hybrid antigen-binding protein having at least two distinct binding sides with different specificities. Bispecific antigen-binding constructs can be produced by a variety of methods including fusion of hybridomas or linking of Fab′ fragments (see, e.g., Songsivilai & Lachmann, Clin. Exp. Immunol. 79:315-321 (1990)).

### Recombinant Cells

The disclosure provides a recombinant cell comprising a deletion of a gene encoding a transporter associated with antigen processing (TAP) protein. The TAP protein complex belongs to the family of ABC transporters, and plays a crucial role in the processing and presentation of the major histocompatibility complex (MHC) class I restricted antigens. The TAP protein complex is comprised of the TAP-1 and TAP-2 proteins, which each have one hydrophobic region and one ATP-binding region. TAP-1 and TAP-2 assemble into a heterodimer, which results in a four-domain transporter. TAP transports peptides from the cytosol into the endoplasmic reticulum, thereby selecting peptides matching in length and sequence to respective MHC class I molecules. Upon loading on MHC class I molecules, the trimeric MHC class I/beta2-microglobulin/peptide complex is then transported to the cell surface and presented to CD8+ cytotoxic T cells. The recombinant cell may comprise a deletion, in whole or in part, of any TAP gene, such that the function of the TAP gene is obliterated or impaired. Not to adhere to any particular theory, a TAP-deficiency may be exploited to facilitate the exogenous loading of peptides onto HLA class I proteins expressed by the recombinant cell.

In some embodiments, the recombinant cell may be a lymphoblastoid cell line (LCL). LCLs typically are generated by Epstein-Barr virus (EBV) transformation of B-lymphocytes within the peripheral blood lymphocyte (PBL) population (Hussain, T. and Mulherkar, R., Int J Mol Cell Med. Spring; 1(2): 75-87 (2012)). Alternatively, lymphoblastoid cell lines may be derived spontaneously from peripheral blood B lymphocytes. A variety of lymphoblastoid cells are known in the art and can be used in the context of the present disclosure. Exemplary lymphoblastoid cell lines include, but are not limited to, T2 (ATCC CRL-1992), K-562 (ATCC CCL-243), and DG75 (ATCC CRL-2625). In some embodiments, the lymphoblastoid cell line may be a hybrid T and B lymphoblastoid cell, such as, for example, a T2 cell. The T2 cell line is an Epstein-Barr Virus-transformed lymphoblastoid (EBV-B) TAP-deficient cell line. As a result, T2 cells mainly express unstable empty HLA class I molecules on their surface (Hosken N A, Bevan M J., Science, 248(4953): 367-70 (1990) doi: 10.1126/science.2326647) and endogenously express very low levels of cell surface HLA-A*02:01 because of an inability to access endogenously processed intracellular peptides. The use of T2 cells to probe antigen recognition by CTLs is well-established. T2 cells also express CD3ε on the cell surface. Upon pulsing T2 cells with an HLA-A*02:01 permissive peptide, HLA-A*02:01 expression on the cell surface increases.

In further aspects, the recombinant cell may comprise at least one genome mutation in a CD3ε gene and at least one genome mutation in an HLA-A gene, such that that the recombinant cell does not express any HLA-A or CD3ε on the cell surface. Not to adhere to any particular theory, mutation of both an HLA-A gene and a CD3 receptor complex gene (e.g., CD3ε) may disrupt the ability of the recombinant cell to present endogenous peptides, allowing for introduction of exogenous HLA-alleles to assess alloreactivity in the presence of target peptide(s). While mutation of an HLA-A gene is desirable, in some embodiments the recombinant cell may comprise at least one mutation in an HLA-B and/or HLA-C gene.

It will be appreciated that human T lymphocytes recognize antigen in the form of peptide bound to major histocompatibility complex molecules (pMHC). MHC molecules generally are highly polymorphic glycoproteins encoded by MHC class I or MHC class II genes. MHC molecules in humans are also designated “human leukocyte antigens (HLAs).” There are two classes of MHC-molecules: MHC class I molecules and MHC class II molecules. MHC molecules are composed of an alpha heavy chain and beta-2-microglobulin (MHC class I receptors) or an alpha and a beta chain (MHC class II receptors), respectively. Their three-dimensional conformation results in a binding groove, which is used for non-covalent interaction with peptides. MHC class I molecules can be found on most cells having a nucleus, and present peptides that result from proteolytic cleavage of predominantly endogenous proteins and larger peptides. MHC class II molecules can be found predominantly on professional antigen presenting cells (APCs), and primarily present peptides of exogenous or transmembrane proteins that are taken up by APCs during the course of endocytosis, and are subsequently processed. Complexes of peptide and MHC class I molecules are recognized by CD8-positive cytotoxic T-lymphocytes bearing the appropriate T-cell receptor (TCR), whereas complexes of peptide and MHC class II molecules are recognized by CD4-positive-helper-T cells bearing the appropriate TCR. It is well known that the TCR, the peptide, and the MHC are present in a stoichiometric amount of 1:1:1.

In humans, the classical HLA loci include class Ia (HLA-A, -B, -C), class Ib (HLA-E, -F, -G, -H), and class II (HLA-DR, -DQ, -DM, and -DP), which are involved in antigen presentation to CD8+ T cells, natural killer cells (NK cells), and CD4+ T cells, respectively. They are encoded in a 3,500 kb segment on human chromosome 6p21.3, which is the most variable region in the human genome (Shiina et al., Journal of Human Genetics, 54: 15-39 (2009)). Human leukocyte antigens are one of the most polymorphic genes in humans, with several thousand alleles encoding for functional polypeptides, enabling the immune system to respond to a diversity of microorganisms and antigens the host encounters. Genotyping for HLA-A polymorphisms is routinely performed for bone marrow and kidney transplantation.

The HLA-A antigens are encoded by the HLA-A locus. More than 7,000 HLA-A alleles have been identified (see, e.g., Holdsworth et al., Tissue Antigens, 73, 95-170 (2008)). HLA-A molecules comprise a heterodimer of a heavy chain and a light chain (beta-2 microglobulin). The heavy chain is anchored in the membrane. The heavy chain is approximately 45 kDa and its gene contains 8 exons. Exon 1 encodes the leader peptide, exons 2 and 3 encode the alpha1 and alpha2 domains, which both bind the peptide, exon 4 encodes the alpha3 domain, exon 5 encodes the transmembrane region, and exons 6 and 7 encode the cytoplasmic tail.

A recombinant cell encompassed by the disclosure may comprise a mutation of any HLA-A allele. HLA-A alleles that occur with high frequency in the human population include, for example, A*02:01:01:01, A*02:02:01:01, A*02:03:01, A*02:05:01:01, A*02:06:01:01, A*02:07:01:01, A*02:11:01:01, A*01:01:01:01, A*03:01:01:01, A*11:01:01:01, A*23:01:01:01, A*24:02:01:01, A*30:01:01:01, A*31:01:02:01, A*33:03:01:01, A*68:01:01:01, A*68:02:01:01, A*69:01:01:01, and A*74:01:01:01, any of which can be mutated in the recombinant cell. Other HLA-A alleles that may be mutated are disclosed in, for example, the IPD-IMGT/HLA Database (Robinson et al., Nucleic Acids Research, 48, Issue D1: D948-D955 (2020); doi.org/10.1093/nar/gkz950; ebi.ac.uk/ipd/imgt/hla/). In some embodiments, the HLA-allele is HLA-A*02:01.

The CD3 complex serves as a T cell co-receptor that associates noncovalently with the T cell receptor (TCR). The CD3 protein complex is composed of four chains: a CD3γ (gamma) chain, a CD3δ (delta) chain, and two CD3γ (epsilon) chains. These chains associate with the T cell receptor (TCR) and the so-called ζ (zeta) chain to form the T cell receptor CD3 complex, leading to the generation of an activation signal in T lymphocytes. The CD3γ, CD3δ, and CD3ε chains are highly related cell-surface proteins of the immunoglobulin superfamily containing a single extracellular immunoglobulin domain. The intracellular tails of the CD3 molecules contain a single conserved motif known as an immunoreceptor tyrosine-based activation motif (ITAM), which is essential for the signaling capacity of the TCR. In humans, the CD3ε polypeptide is encoded by the CD3E gene which resides on chromosome 11.

One or more mutations (e.g., a deletion, insertion, or substitution) of the HLA-A gene and CD3 gene may be generated using any suitable method known in the art. Such methods include, for example, site-directed mutagenesis (SDM) and random-and-extensive mutagenesis (REM), both of which can be performed by PCR and non-PCR-based techniques. In other embodiments, mutations may be introduced using gene editing. Gene editing methods frequently used in the art include, for example, clustered regularly interspaced short palindromic repeats (“CRISPR”) technology, TALENs (transcription activator-like effector nucleases), and Zinc-Finger Nucleases (ZFN), any of which may be used in the context of the present disclosure. In some embodiments, gene editing is performed using a CRISPR system or method. It is understood in the art that the CRISPR system has been exemplified with Cas9 enzymes, but numerous other enzymes have been shown to be suitable for use with CRISPR guide RNAs. As used herein, the term “CRISPR system” refers collectively to transcripts and other elements involved in the expression of and/or directing the activity of CRISPR-associated genes, including nucleotide sequences encoding an enzyme, the same enzyme in protein form, a tracr RNA (trans-activating CRISPR) sequence (e.g., tracrRNA or an active partial tracrRNA), a cr RNA sequence (e.g., crRNA or an active partial crRNA), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Staphylococcus aureus or Streptococcus pyogenes. In certain embodiments, the Cas9 protein can be included in the system separate from, associated with, or encoded by, a vector.

Any element of any suitable CRISPR gene editing system known in the art can be employed, as appropriate. CRISPR gene editing technology is described in detail in, for example, U.S. Pat. Nos. 8,697,359; 8,771,945; 8,945,839; 10,000,772; and U.S. Pat. No. 10,113,167; Cong et al., Science, 339: 819-823 (2013); Makarova et al., Nature Reviews Microbiology, 9(6): 467-477 (2011); Wiedenheft et al., Nature, 482: 331-338 (2012); Gasiunas et al., Proceedings of the National Academy of Sciences USA, 109(39): E2579-E2586 (2012); Jinek et al., Science, 337: 816-821 (2012); Carroll, Molecular Therapy, 20(9): 1658-1660 (2012); Al-Attar et al., Biol Chem., 392(4): 277-289 (2011); aid Hale et al., Molecular Cell, 45(3): 292-302 (2012); Hanna R E, Doench J G., Nat Biotechnol., 38(7): 813-823 (2020). doi: 10.1038/s41587-020-0490-7.; and Janik et al., Int J Mol Sci., 21(24): 9604 (2020). doi: 10.3390/ijms21249604.

The degree to which expression of the HLA-A allele and CD3 gene is inhibited by the one or more mutations may be partially complete (e.g., 10% or more, 25% or more, 50% or more, or 75% or more), substantially complete (e.g., 85% or more, 90% or more, or 95% or more), or fully complete (e.g., 98% or more, or 99% or more). Ideally, the mutations desirably are sufficient to completely inhibit, block, ablate, or “knockout” expression of each gene, such that cell does not produce any HLA-A protein and CD3ε protein, or produces either or both proteins at undetectable levels.

While the recombinant cell described herein does not express an endogenous HLA-A gene, in certain embodiments the cell comprises an exogenous nucleic acid sequence encoding an HLA-A allele. An “exogenous” nucleic acid sequence is any nucleic acid sequence (e.g., DNA, RNA, or cDNA sequence) that is not a naturally occurring nucleic acid sequence present in a cell. The terms “exogenous nucleic acid sequence,” “non-native nucleic acid sequence,” and “heterologous nucleic acid sequence” are synonymous and can be used interchangeably in the context of the disclosure. The exogenous nucleic acid sequence may encode any suitable HLA-A allele, such as those disclosed herein or otherwise known in the art. For example, the exogenous nucleic acid sequence may encode one of the following HLA-A alleles: HLA-A*02:01, HLA-A*02:03, HLA-A*02:05, HLA-A*02:06, or HLA-A*02:07. In embodiments where the recombinant cell comprises a mutation of an HLA-B or HLA-C gene, however, the cell desirably comprises an exogenous nucleic acid sequence encoding an HLA-B allele or HLA-C allele, respectively. HLA-B and HLA-C alleles are known in the art. Indeed, according to the World Health Organization Nomenclature Committee for Factors of the HLA System, there are over 8,000 HLA-B alleles and over 7,000 HLA-C alleles (see, e.g., IPD-IMGT/HLA Database (Robinson et al., Nucleic Acids Research, 48, Issue D1: D948-D955 (2020); doi.org/10.1093/nar/gkz950; ebi.ac.uk/ipd/imgt/hla/).

A nucleic acid sequence encoding an HLA-A allele may be introduced into cells by any suitable method, including, for example, by transfection, transformation, or transduction. The terms “transfection,” “transformation,” and “transduction” are used interchangeably herein and refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); strontium phosphate DNA co-precipitation (Brash et al., Mol. Cell. Biol., 7: 2031-2034 (1987); and magnetic nanoparticle-based gene delivery (Dobson, J., Gene Ther, 13(4): 283-7 (2006)). Phage or viral vectors can be introduced into host cells, after growth of infectious particles in suitable packaging cells, many of which are commercially available.

In some embodiments, a viral vector may be used to introduce an exogenous nucleic acid encoding an HLA-A allele into cells. Suitable viral vectors and methods of preparing same are well known in the art (see, e.g., Miguel Sena-Esteves and Guangping Gao, Cold Spring Harb Protoc; doi:10.1101/pdb.top095513). Representative viral vectors include, but are not limited to, adenovirus-based vectors, adeno-associated virus (AAV)-based vectors, lentivirus-based vectors, and retrovirus-based vectors. In some embodiments, the viral vector is a retroviral vector. Retroviruses are positive-strand RNA viruses that stably integrate their genomes into host cell chromosomes. When pseudotyped with an envelope that has broad tropism, such as vesicular stomatitis virus glycoprotein (VSV-G), retroviruses can enter virtually any mammalian cell type. The capacity of retroviruses for foreign nucleic acid sequences is about 8 kb. Retroviral vectors have been extensively used to deliver therapeutic genes in the context of gene therapy, clinical applications for monogenic disorders, cancer, and infectious diseases, and provide stable and efficient expression of transgenes in humans.

In addition to the exogenous nucleic acid encoding an HLA-A allele, the vector desirably comprises expression control sequences, such as promoters, enhancers, polyadenylation signals, transcription terminators, internal ribosome entry sites (IRES), and the like, that provide for the expression of the coding sequence in a host cell. Exemplary expression control sequences are known in the art and described in, for example, Goeddel, Gene Expression Technology: Methods in Enzymology, Vol. 185, Academic Press, San Diego, Calif (1990); Goldstein et al., Biotechnology Annual Review, Elsevier, Volume 1, 1995, Pages 105-128; doi.org/10.1016/S1387-2656(08)70049-8; Ho S C, Yang Y., Biotechnol Lett., 36(8):1569-79 (2014). doi: 10.1007/s10529-014-1523-4; and Green and Sambrook, Molecular Cloning, a Laboratory Manual, 4^{th} edition, Cold Spring Harbor Press, Cold Spring Harbor, N.Y. (2012).

### Libraries and Systems

In some embodiments, a plurality of the disclosed recombinant cells are engineered to express different exogenous HLA-A alleles, so as to produce a library of recombinant cells, wherein each cell comprises an exogenous nucleic acid sequence encoding a different HLA-A allele. As used herein, the term “library” refers to a plurality of polynucleotides, proteins, or cells comprising a collection of two, or two or more, non-identical but related members. The term “expression library” refers to a collection of vectors each containing a different nucleic acid sequence (e.g., cDNA, genomic DNA) that are constructed in such a way that they will be transcribed and translated by a host organism or cell. Methods for generating expression libraries using a variety of different vectors and host cells are known in the art (see, e.g., U.S. Pat. No. 8,603,950, Yang et al., Nat Methods, 8: 659-661 (2011). doi.org/10.1038/nmeth.1638; and Lodes et al., Methods Mol Med., 94: 91-106 (2004)) and any such method may be used to generate the libraries described herein. For example, individual DNA library members, each of which encodes a peptide or protein to be expressed, may be introduced into a suitable vector (e.g., plasmid, phage, or virus). The vector into which the DNA library member is introduced includes all the sequences necessary (e.g., promoters, and translation start and stop signals) to allow expression of the peptide or protein from the vector. The number of distinct members of the library will depend on a number of factors, such as the number of exogenous nucleic acid sequences encoding different HLA-A alleles. In some embodiments, the library may comprise at least 10⁴ discrete exogenous nucleic acid sequences. For example, the library may contain more than 10⁶, more than 10⁸, more than 10¹⁰, more than 10¹², or more than 10¹⁴, discrete exogenous nucleic acid sequences.

The disclosure further provides a system comprising the above-described library of recombinant cells in combination with one or more peptides, one or more immunotherapeutic agents, and one or more T cells. Many proteins and peptides are known to be expressed at high levels on cancer cells, on cells that mediate an autoimmune or inflammatory condition, or on infectious agents or cells infected thereby. Such proteins and peptides are potential target molecules for immunotherapeutics, such as monoclonal and bispecific antibodies, engineered T cells (e.g., CAR-T cells), engineered T cell receptors (TCRs), and bispecific T cell engager (BiTE®) molecules. Thus, the one or more peptides included in the disclosed system desirably are based on or derived from a protein or peptide that can be targeted by an immunotherapeutic agent.

In this regard, each of the one or more peptides desirably is an antigen, preferably a peptide antigen. In some embodiments, the one or more peptides may be based on or derived from a peptide or protein that is expressed on human cancer cells. In such cases, the peptide antigen may be referred to herein as a “cancer antigen” or “tumor antigen.” Cancer antigens include, but are not limited to, WT1, MUC1, LMP2, EGFRvIII, HER-2/neu, MAGE-A3, NY-ESO-1, PSMA, synthase, CEA, MLANA/MART1, gp100, survivin, prostate-specific antigen (PSA), telomerase reverse transcriptase (hTERT), sarcoma translocation breakpoints, EPHA2, prostatic acid phosphatase (PAP), melanoma inhibitor of apoptosis (ML-IAP), α-fetoprotein (AFP), epithelial cell adhesion molecule (EpCAM), ERG, NA17.A2 peptide (VLPDVFIRC), paired box 3 (PAX3), anaplastic lymphoma kinase (ALK), androgen receptor, cyclin B1, polysialic acid, rho-related GTP-binding protein RhoC, v-myc myelocytomatosis viral related oncogene (MYCN), TRP-2, GD3 ganglioside, fucosyl GM1, mesothelin, prostate stem cell antigen (PSCA), MAGE-A1, CYP1B1, PLAC1, GM3, BORIS, tetranectin (TN), ETV6-AML1 (especially peptides including the breakpoint), NY-BR-1, RGS5, SART3, STn, carbonic anhydrase IX, PAX5, proacrosin binding protein sp32 precursor (OY-TES-1), sperm protein 17 (Sp17), LCK, high molecular weight melanoma-associated antigen (HMWMAA, also known as melanoma chondroitin sulfate proteoglycan), AKAP-4, SSX2, XAGE-1, B7H3 (also known as CD276), legumain, TIE2, prostate-associated gene 4 protein (PAGE-4), vascular endothelial growth factor receptor 2 (VEGFR2), protamine 2 (also known as MAD-CT-1), glomulin (also known as FAP), PDGFR-I3, SSX2, SSX5, Fos-related antigen 1, CD20, integrin avP3, 5T4 oncofetal antigen, CA IX, CDS, CD19, CD22 (also known as Siglec-2), CD30 (also known as TNFRSF1), CD33 (also known as Siglec-3), CD40, CD44V6, CD55, CD56 (also known as NCAM), (also known as CD152), EGFR, GD2, HER2, HLA-DR10 (MHC II), IGF1R, IL-6, sialyl Lewis Y, TAG-72, TAL6, TRAILR2, VEGF, CD52 (also known as CAMPATH-1), CD4, CD73, CA125 (also known as MUC16), CD66e, CD80 (also known as B7-1), PDGFRI3, B cell maturation antigen (BCMA), DLL3, FLT3, MUC17, CLDN18.2, and prostate specific membrane antigen (PSMA, also known as glutamate carboxypeptidase 2, among many other names).

Cancer antigens also include the human herpes virus 4 protein LMP2, the human papillomavirus proteins E6 and E7, and the glycoceramide globo H (as described in Gilewski et al. (2001), Proc. Natl. Acad. Sci., 98(6): 3270-3275), the a4 subunit of the a4131 and a4137 integrins, the a4137 integrin, BAFF, APRIL, CD2, CD3, CD20, CD52, CD73, CD80, CD86, the C5 complement protein, IgE, IL-113, IL-5, IL-6R, IL-12, IL-23, and tumor necrosis factor alpha (TNF-α).

In some embodiments, the one or more peptides included in the system may be a MAGE peptide, a BCMA peptide, a CD19 peptide, a CD33 peptide, a DLL3 peptide, a FLT3 peptide, a MUC17 peptide, a PSMA peptide, or a CLDN18.2 peptide. It will be appreciated that selection of the one or more peptides will largely be dependent on the specificity of the immunotherapeutic agent (e.g., BiTE© molecule or engineered TCR) being interrogated and the primary sequence of the target peptide. Methods for selecting an appropriate peptide are well known in the art. In some embodiments, peptide selection may be accomplished using a bioinformatic pipeline that predicts peptides having the highest likelihood of simultaneously (a) binding to the HLA class I allotype of interest and (b) being recognized by the immunotherapeutic agent.

The system further comprises one or more immunotherapeutic agents. The term “immunotherapeutic agent,” as used herein, refers to any substance, compound, molecule, or modality that triggers a patient's immune system to fight a particular disease, typically cancer. Immunotherapeutics generally rely on the use of immune effector cells and molecules to target and destroy cancer cells. The immune effector may be, for example, an antibody or antibody fragment specific for some marker on the surface of a tumor cell. The antibody or antibody fragment alone may serve as an effector of therapy or it may recruit other cells to actually effect cell killing. The antibody or antibody fragment may also prevent cancer immunoevasion or immunosuppression. The antibody or antibody fragment also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include, for example, cytotoxic T cells, NKT cells, and NK cells.

T cells or T lymphocytes are a type of lymphocyte (itself a type of white blood cell) that play a central role in cell-mediated immunity. There are several subsets of T cells, each with a distinct function. T cells can be distinguished from other lymphocytes, such as B cells and NK cells, by the presence of a T cell receptor (TCR) on the cell surface. The TCR is responsible for recognizing antigens bound to major histocompatibility complex (MHC) molecules and is composed of two different protein chains. In 95% of human T cells, the TCR consists of an alpha (a) and beta (s) chain. When the TCR engages with an antigenic peptide and MHC (peptide/MHC complex), the T lymphocyte is activated through a series of biochemical events mediated by associated enzymes, co-receptors, specialized adaptor molecules, and activated or released transcription factors.

In some embodiments, the immunotherapeutic agent comprises a T cell-based therapeutic. In recent years, genetically modified T cells have been used in adoptive T cell therapy (ATC), in which autologous or allogeneic T cells are infused into a cancer patient. For example, some ATC methodologies involve extracting T cells from a subject, genetically modifying the T cells (e.g., to express a desired construct (e.g., a chimeric antigen receptor (CAR) or engineered TCR)), cultured in vitro, and returned to the subject. In “autologous” administration methods, cells (e.g., lymphocytes) are removed from a mammal, stored, engineered or modified and returned back to the same mammal. In “allogeneic” administration methods, a mammal receives cells (e.g., blood-forming stem cells or lymphocytes) from a genetically similar, but not identical, donor. Ideally, the cells are autologous to the mammal.

Two main types of engineered T cells are those that contain chimeric antigen receptors (termed “CARs” or “CAR-Ts”) or engineered T-cell receptors (“TCRs”). These engineered T cells are genetically modified to endow them with antigen specificity while retaining or enhancing their ability to recognize and kill a target cell. Chimeric antigen receptors may comprise, for example, (i) an antigen-specific component (“antigen-binding molecule”), (ii) one or more costimulatory domains, and (iii) one or more activating domains. Each domain may be heterogeneous, that is, comprised of sequences derived from different protein chains. Chimeric antigen receptor-expressing immune cells (such as T cells) may be used in various therapies, including cancer therapies. It will be appreciated that costimulating polypeptides as defined herein may be used to enhance the activation of CAR-expressing cells against target antigens, and therefore increase the potency of adoptive immunotherapy. T cells can be engineered to possess specificity to one or more desired targets. For example, T cells can be transduced with DNA or other genetic material encoding an antigen-binding molecule, such as one or more single chain variable fragment (“scFv”) of an antibody, in conjunction with one or more signaling molecules, and/or one or more activating domains, such as CD3 zeta.

Unlike CAR-T cells, T cells engineered to express T cell receptors (TCR) specific to a particular tumor antigen (“TCR-T cells”) can exploit a broader range of targets for multiple cancer indications because TCR-T cells can recognize the peptide-MHC complexes (pMHC) derived from intracellular proteins constituting ˜75% of the genome. Bioengineering of the TCR α- and β-glycoprotein antigen-binding domains, while preserving the conserved domains (Cα and Cβ), allows for the development and expansion of T lymphocytes with specificity to MHC-associated tumor neoantigens.

In some embodiments, the immunotherapeutic agent comprises an engineered T cell receptor (TCR) or a bispecific antigen-binding molecule, such as a BiTE® (bispecific T cell engager) molecule. Bispecific antigen-binding molecules such as BiTE® molecules are recombinant protein constructs made from two flexibly linked antibody derived binding domains. One binding domain of BiTE® molecules is specific for a selected tumor-associated surface antigen on target cells; the second binding domain is specific for CD3. By their particular design, BiTE® molecules are uniquely suited to transiently connect T cells with target cells and, at the same time, potently activate the inherent cytolytic potential of T cells against target cells (see, e.g., WO 99/54440, WO 2005/040220, and WO 2008/119567).

As discussed above, immunotherapeutics generally rely on the use of immune effector cells to target and destroy cancer cells. Thus, the system further comprises one or more effector cells, such as cytotoxic T cells, NKT cells, and NK cells. In some embodiments, the system comprises effector T cells. “Effector” lymphocytes, such as T cells, can mediate the removal of tumor cells or pathogens without the need for further differentiation. Effector lymphocytes are distinct from naïve lymphocytes, which must proliferate and differentiate before mediating effector functions, and memory lymphocytes, which must differentiate and often proliferate before becoming effector cells. Effector T cells may be classified as CD8 cytotoxic cells, T_{H}1 cells, or T_{H}2 cells. CD8 cytotoxic cells kill target cells that display peptide fragments of cytosolic pathogens bound to MHC class I molecules at the cell surface. T_{H}1 and T_{H}2 cells both express the CD4 coreceptor and recognize fragments of antigens degraded within intracellular vesicles, displayed at the cell surface by MHC class II molecules. T_{H}1 cells activate macrophages, while T_{H}2 cells drive B cell differentiation and immunoglobulin production.

### Methods

In some embodiments, the disclosed system may be used to screen for alloreactivity of a immunotherapeutic agent of interest. Thus, the disclosure also provides a method for determining alloreactivity of an immunotherapeutic agent, which method comprises: (a) contacting the above-described library of recombinant cells of with one or more peptides, one or more immunotherapeutic agents, and one or more T cells, whereby the one or more peptides are presented at the surface of the recombinant cell bound to one or more major histocompatibility complex (MHC) molecules (pMHC); and (b) assessing cytotoxicity of the recombinant cells, wherein increased cytotoxicity as compared to control cells indicates alloreactivity of the immunotherapeutic agent.

The disclosed system also may be used to assess binding specificity of the immunotherapeutic agent to a peptide presented at the surface of the recombinant cells. Thus, the disclosure also provides a method for determining binding specificity between a peptide and an immunotherapeutic agent, which method comprises: (a) contacting the above-described library of recombinant cells with one or more peptides, one or more immunotherapeutic agents, and one or more T cells, whereby the one or more peptides are presented at the surface of the recombinant cell bound to one or more major histocompatibility complex (MHC) molecules (pMHC); and (b) assessing cytotoxicity of the recombinant cells, wherein increased cytotoxicity as compared to control cells indicates that the immunotherapeutic agent specifically binds to the peptide. Descriptions of the peptides, immunotherapeutic agents, and T cells set forth herein in connection with the aforementioned cells and system also are applicable to those same aspects of the disclosed methods.

An agent is “cytotoxic” and induces “cytotoxicity” if the agent (e.g., an immunotherapeutic) kills, induces killing, or inhibits the growth of cells (e.g., cancer cells). With respect to cancer cells, for example, cytotoxicity includes preventing cancer cell division and growth, as well as reducing the size of a tumor or cancer. Cytotoxicity of the recombinant cells displaying a peptide-MHC complex at the cell surface may be measured using any suitable cell viability assay known in the art. Such assays include, but are not limited to, assays which measure cell lysis, cell membrane leakage, and apoptosis. For example, methods including, but not limited to, trypan blue assays, propidium iodide assays, lactate dehydrogenase (LDH) assays, tetrazolium reduction assays, resazurin reduction assays, protease marker assays, 5-bromo-2′-deoxy-uridine (BrdU) assays, and ATP detection may be used. Cell viability assay systems that are commercially available also may be used and include, for example, CELLTITER-GLO® 2.0 (Promega, Madison, WI), VIVAFIX™ 583/603 Cell Viability Assay (Bio-Rad, Hercules, CA); and CYTOTOX-FLUOR™ Cytotoxicity Assay (Promega, Madison, WI). In some embodiments, cytotoxicity is assessed by performing a T cell dependent cellular cytotoxicity (TDCC) assay. TDCC assays measure the percentage of specific cytotoxicity induced when an immunotherapeutic agent (e.g., a BiTE© molecule or engineered TCR) engages T-cells, redirects T-cell mediated cytolysis, and ultimately kills target cells. In some embodiments, the TDCC assay is a luciferase-based (also referred to as “luminescence-based”) assay. Luminescence-based TDCC assays can quantify live target cells through the detection of constitutively expressed luciferase. Luminescence readout is a common approach for quantitative cell-based assays that exhibits good sensitivity and robustness. Firefly luciferase has a short half-life of a few hours in cells and rapidly decays in assay media. In the context of the present disclosure, the recombinant cells (e.g., T2 cells) are stably transduced to express firefly luciferase. T-cells are not modified and do not provide luminescence to the assay, so the remaining live target cells can be quantified. TDCC assays are further described in, e.g., Nazarian et al., J. Biomolecular Screening, 20(4): 519-527 (2015); Fu et al., PLoS ONE, 5(7): e11867, 1-6 (2010); Schafer et al., J. Immunol. Methods., 204(1): 89-98 (1997); and Ross et al., PLoS ONE, 12(8): e0183390 (2017).

It will be appreciated that cytotoxicity of the recombinant cells expressing an exogenous HLA-A allele may be assessed in comparison to appropriate control cells. In some embodiments, control cells comprise at least one mutation in a CD3ε gene and at least one mutation in an HLA-A gene, but lack an exogenous nucleic acid sequence encoding an HLA-A allele. That is, the control cells may be identical to the recombinant cells provided herein, except for the presence of the exogenous HLA-A allele. For example, the control cells may be T2 cells comprising a double knockout of the CD3ε gene and an HLA-A allele and lacking an exogenous HLA-A coding sequence. In some embodiments, the control cells also express a luciferase gene. As described above, increased cytotoxicity of the recombinant cells as compared to control cells induced by immunotherapeutic agent (e.g., a BiTER molecule or engineered TCR) recognition of recombinant cells transduced with a non-target HLA-A allele loaded with an non-target peptide indicates alloreactivity of the immunotherapeutic agent.

The following examples further illustrate the invention but, of course, should not be construed as in any way limiting its scope.

### Example 1

This example describes the production of a T2 cell comprising a double knockout of the CD3ε gene and the HLA-A*02:01 allele.

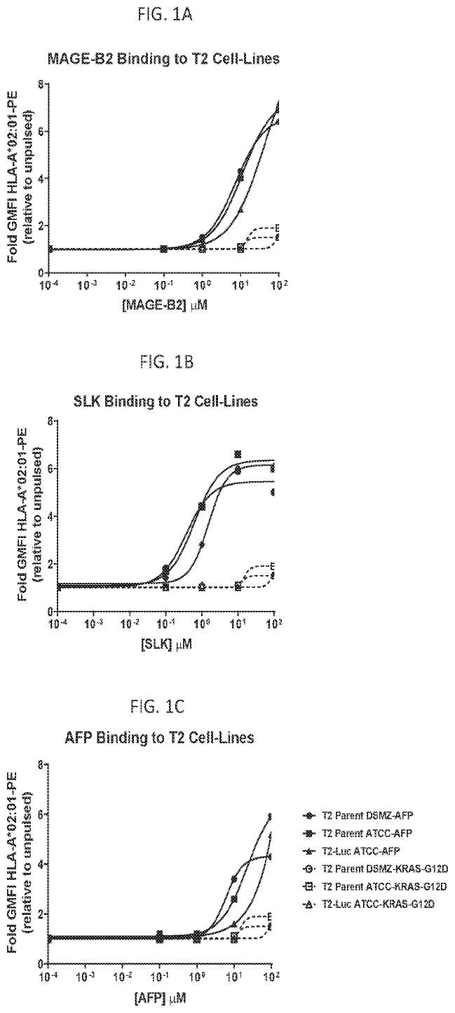

Parental T2 cells expressing luciferase (T2-Luc) were obtained and shown to stabilize HLA-A*02:01 in a dose-responsive matter with different peptides, as shown in FIGS. 1A-1C.

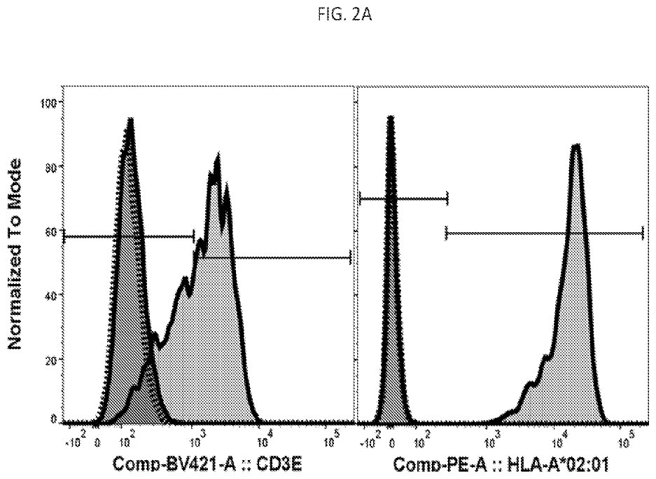

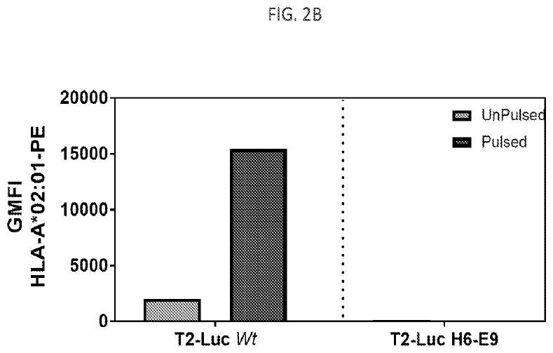

A parental T2 cell line that does not express any HLA-A or CD3ε on the cell surface was generated by knocking out HLA-A*02:01 and CD3ε. FACS analysis using commercially available antibodies was performed to assess HLA-A*02:01 and CD3ε protein expression at the cell surface. Transfected cell cultures were expanded, and cells harboring the double knockout were bulk-sorted. HLA allotyping was performed to confirm deletion of base pairs in the targeted region of HLA-A*02:01 at the genomic level. FIG. 2A demonstrates the successful knockout of CD3ε and HLA-A*02:01, as confirmed at the cell surface protein level. Confirmation of successful HLA-A*02:01 knockout was further confirmed when wild type T2 and HLA-A*02 knockout cells were pulsed with peptide and no increase in cell surface HLA-A*02 was observed when compared to the wild type control, as shown in FIG. 2B. These cells were cloned at the single cell level and monoclonal cell lines were generated.

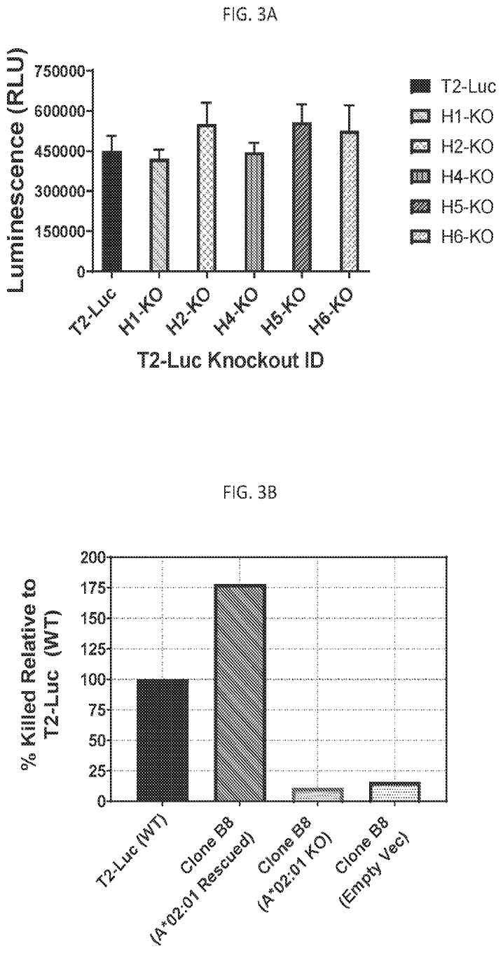

The knockout of CD3ε and HLA-A*02:01 did not affect luciferase expression in the T2 cells (see FIG. 3A). To determine that the HLA class I antigen processing and presentation machinery remained intact after knockout of HLA-A*02:01 and CD3ε, a rescue experiment was performed whereby HLA-A*02:01 was re-introduced into the modified T2 cells by retroviral transduction. Specifically, the rescue experiment was performed using a pMIG-GFP or pMIG-HLA-A*02:01-B2M-GFP retroviral construct to transduce T2-Luc cells, and transduced cells were sorted for GFP expression. After 72 hours in culture, these cells were pulsed with MAGE-B2 peptide co-cultured with TCR-T cells specific to HLA-A*02:01 molecules presenting the MAGE-B2 peptide (Donor 8400 X6-013). As shown in FIG. 3B, the HLA-A*02:01/CD3ε knockout T2 cell line pulsed with MAGE-B2 peptide was recognized and killed by specific TCR-T cells when compared to empty vector control or peptide unpulsed knockout cells.

These results demonstrate that the HLA class I antigen processing and presentation pathway remains functional in HLA-A*02:01/CD3ε double knockout T2 cells.

### Example 2

This example describes the generation of an alloreactivity screening system using the double knockout T2 cells described in Example 1.

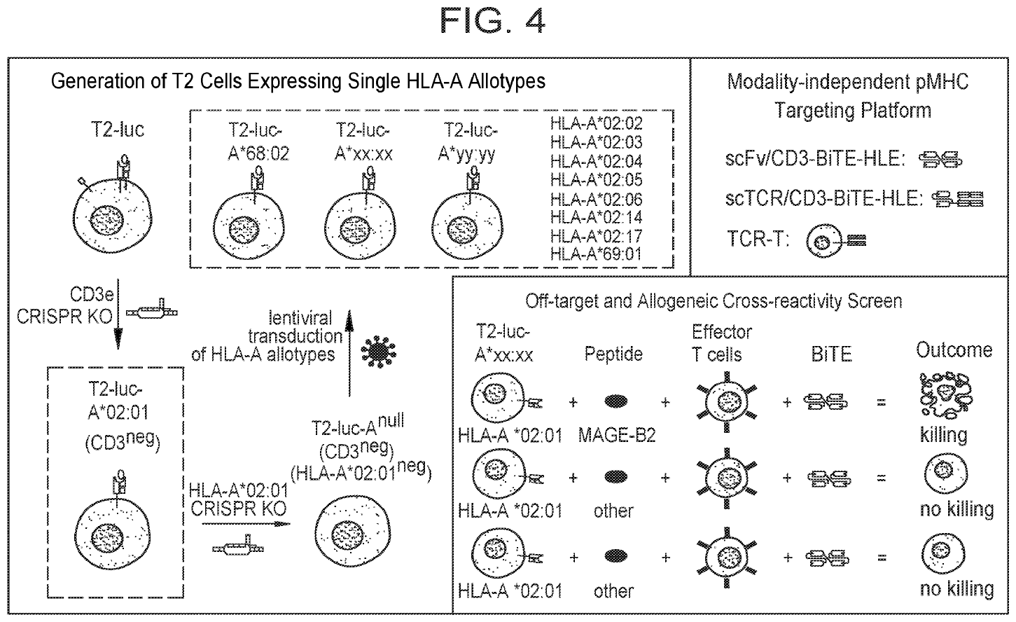

As shown schematically in FIG. 4, the recombinant cells described herein provide for a controlled system to test both alloreactivity and specificity of immunotherapeutic agents. In this regard, for example, the HLA-A*02:01/CD3ε double knockout T2 cells described above can be engineered to express one specific HLA-A cell surface allotype of interest, and target therapeutic peptides can be chosen based both upon target and likelihood of binding to the specific HLA. The system also may be used to evaluate specificity of a TCR (e.g., off-target testing), and to identify and validate new pHLA targets.

More specifically, HLA-A allotypes of interest were synthesized and cloned into an expression vector ready for retroviral packaging. The HLA-A allotypes were selected based on the highest frequency HLA-A allotypes in the Caucasian population, 19 of which are shown in Table 1.

TABLE 1
High frequency HLA-A Allotypes Selected
for the Generation of T2 library

A*02:01:01:01
A*02:02:01:01
A*02:03:01
A*02:05:01:01
A*02:06:01:01
A*02:07:01:01
A*02:11:01:01
A*01:01:01:01
A*03:01:01:01
A*11:01:01:01
A*23:01:01:01
A*24:02:01:01
A*30:01:01:01
A*31:01:02:01
A*33:03:01:01
A*68:01:01:01
A*68:02:01:01
A*69:01:01:01
A*74:01:01:01

Retroviral vectors were constructed to generate stable T2 cell lines comprising the HLA-A*02:01/CD3ε double knockout described above and expressing a single exogenous HLA-A allotype on the cell surface.

A screening library containing HLA-A*02:01/CD3ε double knockout T2 cells expressing HLA-A allotypes of interest was generated, Briefly, next generation sequencing (NGS) analysis for CD3ε and A*02:01 KO/HLA typing was performed to select a final T2-Luc dual KO clone. The T2-Luc dual KO selected clone was transduced with retroviral particles and incubated for 72 hours. Initial evaluation for surface HLA protein was performed via FACS.

Puromycin selection was initiated 72 hours post transduction for one week, and surface HLA levels were evaluated post selection. Transduced T2-Luc cells were evaluated for surface expression of specific HLA molecules via FACS, and luciferase activity levels were evaluated to ensure high levels of activity. Cells were expanded and cryopreserved for further analysis.

Expanded T2-Luc cells for each allotype were bulk sorted via FACS, and HLA typing was performed via NGS. Positive control peptides were used to pulse each T2-Luc cell-line to evaluate the HLA binding and peptide presentation capability. Bulk Sorted T2-Luc cells lines were expanded and cryopreserved for downstream use.

Cross-reactivity of a candidate TCR-T cell was tested in HLA-A*02:01/CD3ε double KO T2 cells expressing HLA-A alleles HLA-A*02:01, HLA-A*02:03, HLA-A*02:05, HLA-A*02:06, or HLA-A*02:07 loaded with MAGE-B2 peptide. The TCR-T cell exhibited activity against HLA-A*02:01, HLA-A*02:05, and HLA-A*02:06 alleles presenting MAGE-B2 peptide, as measured by TCR-T cell dependent lysis of HLA-A*02 expressing T2 cells.

Cross-reactivity of candidate and BiTE® molecules was tested in HLA-A*02:01/CD3ε double KO T2 cells expressing HLA-A alleles HLA-A*02:01, HLA-A*02:03, HLA-A*02:05, HLA-A*02:06, or HLA-A*02:07 loaded with MAGE-B2 peptide. The BiTE® molecules exhibited activity against HLA-A*02:01. HLA-A*02:03, HLA-A*02:05, HLA-A*02:06, or HLA-A*02:07 presenting MAGE-B2 peptide, as measured by BiTE molecule-mediated lysis of HLA-A*02 expressing T2 cells.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A recombinant cell comprising:
(a) a deletion of a gene encoding a transporter associated with antigen processing (TAP) protein;
(b) at least one genome mutation in a CD3ε gene; and
(c) at least one genome mutation in an HLA-A gene.

2. The recombinant cell of claim 1, which does not express a TAP gene, the CD38 gene, and the HLA-A gene.

3. The recombinant cell of claim 1, wherein the HLA-A gene comprises an HLA-A*02:01 allele.

4. The recombinant cell of claim 1, which is a hybrid T and B lymphoblastoid cell.

5. The recombinant cell of claim 4, which is a T2 cell.

6. The recombinant cell of claim 1, which expresses a luciferase gene.

7. The recombinant cell of claim 1, which comprises an exogenous nucleic acid sequence encoding an HLA-A allele.

8. The recombinant cell of claim 7, wherein the HLA-A allele is A*02:01:01:01, A*02:02:01:01, A*02:03:01, A*02:05:01:01, A*02:06:01:01, A*02:07:01:01, A*02:11:01:01, A*01:01:01:01, A*03:01:01:01, A*11:01:01:01, A*23:01:01:01, A*24:02:01:01, A*30:01:01:01, A*31:01:02:01, A*33:03:01:01, A*68:01:01:01, A*68:02:01:01, A*69:01:01:01, or A*74:01:01:01.

9. A library comprising a plurality of recombinant cells according to claim 7, wherein each cell comprises an exogenous nucleic acid sequence encoding a different HLA-A allele.

10. A system comprising:
(a) the library of recombinant cells of claim 9;
(b) one or more peptides;
(c) one or more immunotherapeutic agents; and
(d) one or more T cells.

11. The system of claim 10, wherein the one or more peptides comprise one or more cancer antigens.

12. The system of claim 11, wherein the one or more peptides is a MAGE peptide, a BCMA peptide, a CD19 peptide, a CD33 peptide, a DLL3 peptide, a FLT3 peptide, a MUC17 peptide, a PSMA peptide, or a CLDN18.2 peptide.

13. The system of claim 12, wherein the peptide is a MAGE peptide.

14. The system of claim 10, wherein the one or more immunotherapeutic agents comprise an engineered T cell receptor (TCR) or a bi-specific T cell engager protein.

15. The system of claim 10, wherein the T cells are effector T cells.

16. A method for determining alloreactivity of an immunotherapeutic agent, which method comprises:
(a) contacting the library of recombinant cells of claim 9 with one or more peptides, one or more immunotherapeutic agents, and one or more T cells, whereby the one or more peptides are presented at the surface of the recombinant cell bound to one or more major histocompatibility complex (MHC) molecules (pMHC); and
(b) assessing cytotoxicity of the recombinant cells, wherein increased cytotoxicity as compared to control cells indicates alloreactivity of the immunotherapeutic agent.

17. A method for determining binding specificity between a peptide and an immunotherapeutic agent, which method comprises:
(a) contacting the library of recombinant cells of claim 9 with one or more peptides, one or more immunotherapeutic agents, and one or more T cells, whereby the one or more peptides are presented at the surface of the recombinant cell bound to one or more major histocompatibility complex (MHC) molecules (pMHC); and
(b) assessing cytotoxicity of the recombinant cells, wherein increased cytotoxicity as compared to control cells indicates that the immunotherapeutic agent specifically binds to the peptide.

18. The method of claim 16, wherein the one or more peptides comprise one or more tumor antigens.

19. The method of claim 18, wherein the one or more peptides is a MAGE peptide, a BCMA peptide, a CD19 peptide, a CD33 peptide, a DLL3 peptide, a FLT3 peptide, a MUC17 peptide, a PSMA peptide, or a CLDN18.2 peptide.

20. The method of claim 19, wherein the peptide is a MAGE peptide.

21. The method of claim 16, wherein the immunotherapeutic agent comprises an engineered T cell receptor (TCR) or a bi-specific T cell engager.

22. The method of claim 16, wherein the T cells are effector T cells.

23. The method of claim 16, wherein the control cells comprise at least one mutation in a CD3ε gene and at least one mutation in an HLA-A gene and lack an exogenous nucleic acid sequence encoding an HLA-A allele.

24. The method of claim 23, wherein the control cells are T2 cells.

25. The method of claim 23, wherein the control cells express a luciferase gene.

26. The method of claim 16, wherein cytotoxicity is assessed by performing a T cell dependent cellular cytotoxicity (TDCC) assay.

27. The method of claim 26, wherein the TDCC assay is a luciferase-based assay.
